(19) United States
(12) Patent Application Publication
(10) Pub. No.: US 2025/0171448 A1
(43) Pub. Date: May 29, 2025

(54) SOLID STATE FORMS OF GUSACITINIB

(71) Applicant: TEVA PHARMACEUTICALS INTERNATIONAL GMBH, Jona (CH)

(72) Inventors: Anantha Rajmohan Muthusamy, Sivakasi (IN); Amit Singh, Greater Noida (IN); Chanchai Singh, Greater Noida (IN); Prathap Rengaraj, Madurai (IN)

(21) Appl. No.: 18/839,779

(22) PCT Filed: Feb. 23, 2023
(86) PCT No.: PCT/US2023/013668

§ 371 Date: Aug. 20, 2024

(30) Foreign Application Priority Data
Feb. 23, 2022 (IN) 20221109506
May 16, 2022 (IN) 202211028117
Jun. 3, 2022 (IN) 202211031922
Sep. 21, 2022 (IN) 202211053997
Feb. 2, 2023 (IN) 202211006675
Feb. 6, 2023 (IN) 202211007408

(51) Int. Cl. C07D 487/04; A61K 31/519

(52) U.S. Cl. CPC C07D 487/04; A61K 31/519

(57) ABSTRACT

The present disclosure encompasses solid state forms of Gusacitinib, in embodiments crystalline polymorphs of Gusacitinib, processes for preparation thereof, and pharmaceutical compositions thereof.

### FIELD OF THE DISCLOSURE

The present disclosure encompasses solid state forms of Gusacitinib, in embodiments crystalline polymorphs of Gusacitinib, processes for preparation thereof, and pharmaceutical compositions thereof.

### BACKGROUND OF THE DISCLOSURE

Gusacitinib, 2-[1-[4-[4-(4-hydroxypiperidin-1-yl)anilino]-5-oxo-6H-pyrimido[4,5-d]pyridazin-2-yl]piperidin-4-yl]acetonitrile, has the following chemical structure:

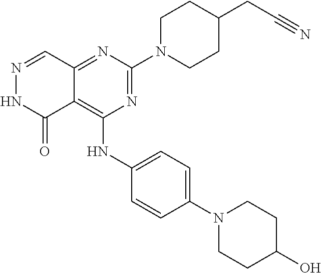 [SMILES: N#CCC1CCN(c2nc(Nc3ccc(N4CCC(O)CC4)cc3)c3c(=O)[nH]ncc3n2)CC1]

Gusacitinib, an oral dual inhibitor of Janus kinase and spleen tyrosine kinase, is under development for the treatment of moderate to severe chronic hand eczema. Gusacitinib is also under clinical investigation for the treatment of moderate to severe atopic dermatitis.

The compound is disclosed in U.S. Pat. No. 8,729,079.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Gusacitinib.

### SUMMARY OF THE DISCLOSURE

The present disclosure provides crystalline polymorphs of Gusacitinib, processes for preparation thereof, and pharmaceutical compositions thereof. These crystalline polymorphs can be used to prepare other forms of Gusacitinib or of Gusacitinib salts.

The present disclosure provides crystalline polymorphs of Gusacitinib for use in the preparation of pharmaceutical compositions and/or formulations for use in medicine, in embodiment for treating dermatological/inflammatory diseases. In particular, for the treatment of moderate to severe chronic hand eczema, or moderate to severe atopic dermatitis, particularly for the treatment of moderate to severe chronic hand eczema.

The present disclosure provides crystalline polymorphs of Gusacitinib for use in medicine, including for treating dermatological/inflammatory diseases, particularly for the treatment of the treatment of moderate to severe chronic hand eczema or moderate to severe atopic dermatitis, and especially for the treatment of moderate to severe chronic hand eczema.

The present disclosure also encompasses the use of crystalline polymorphs of Gusacitinib of the present disclosure for the preparation of pharmaceutical compositions and/or formulations.

In another aspect, the present disclosure provides pharmaceutical compositions comprising any one or a combination of the crystalline polymorphs of Gusacitinib according to the present disclosure.

The present disclosure includes processes for preparing the above mentioned pharmaceutical compositions. The processes include combining any one or a combination of the crystalline polymorphs of Gusacitinib with at least one pharmaceutically acceptable excipient.

The crystalline polymorphs of Gusacitinib as defined herein and the pharmaceutical compositions or formulations of the crystalline polymorphs of Gusacitinib may be used as medicaments, such as for the treatment of dermatological/inflammatory diseases. In particular; for the treatment of moderate to severe chronic hand eczema, or moderate to severe atopic dermatitis, and particularly for the treatment of moderate to severe chronic hand eczema.

The present disclosure also provides methods of treating dermatological diseases by administering a therapeutically effective amount of any one or a combination of the crystalline polymorphs of Gusacitinib of the present disclosure, or at least one of the above pharmaceutical compositions, to a subject suffering from dermatological diseases, or otherwise in need of the treatment.

The present disclosure also provides uses of crystalline polymorphs of Gusacitinib of the present disclosure, or at least one of the above pharmaceutical compositions, for the manufacture of medicaments for treating dermatological/inflammatory diseases. In particular; for the treatment of moderate to severe chronic hand eczema, or moderate to severe atopic dermatitis, and particularly for the treatment of moderate to severe chronic hand eczema.

### BRIEF DESCRIPTION OF THE DRAWINGS

FIG. 1 shows a characteristic X-ray powder diffraction pattern (XRPD) of Gusacitinib-Form GS1.

FIG. 2 shows a characteristic XRPD of Gusacitinib-Form GS2.

FIG. 3 shows a characteristic XRPD of Gusacitinib-Form GS3.

FIG. 4 shows a characteristic XRPD of Gusacitinib-Form GS4.

FIG. 5 shows a characteristic XRPD of Gusacitinib-Form GS5.

FIG. 6 shows a characteristic XRPD of Gusacitinib-Form GS6.

FIG. 7 shows a characteristic XRPD of Gusacitinib-Form GS7.

FIG. 8 shows a characteristic XRPD of Gusacitinib-Form GS8.

FIG. 9 shows a characteristic XRPD of Gusacitinib-Form GS9.

FIG. 10 shows a characteristic XRPD of Gusacitinib HCl salt-Form GHC11.

FIG. 11 shows a characteristic XRPD of Gusacitinib HCl salt-Form GHC12.

FIG. 12 shows a characteristic XRPD of Gusacitinib HCl salt-Form GHC13.

FIG. 13 shows a characteristic XRPD of Gusacitinib HCl salt-Form GHC14.

FIG. 14 shows a characteristic XRPD of Gusacitinib HCl salt-Form GHC15.

FIG. 15 shows a characteristic XRPD of Gusacitinib HCl salt-Form GHC16.

FIG. 16 shows a characteristic XRPD of Gusacitinib HCl salt-Form GHC17.

FIG. 17 shows a characteristic XRPD of Gusacitinib HCl salt-Form GHC18.

### DETAILED DESCRIPTION OF THE DISCLOSURE

The present disclosure encompasses solid state forms of Gusacitinib, processes for preparation thereof, and pharmaceutical compositions thereof.

The solid state or polymorph forms of Gusacitinib or Gusacitinib HCl as described in any aspect or embodiment of the disclosure may be polymorphically pure or substantially free of any other forms. A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, any of the solid state forms of Gusacitinib HCl as described herein may be substantially free of any other solid state forms of Gusacitinib HCl. Similarly any of the solid state forms of Gusacitinib as described herein may be substantially free of any other solid state forms of Gusacitinib. Thus, a crystalline polymorph of Gusacitinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Gusacitinib. Similarly, a crystalline polymorph of Gusacitinib HCl according to any aspect or embodiment of the present disclosure which is polymorphically pure or substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Gusacitinib HCl. In some embodiments of the disclosure, the described crystalline polymorph of Gusacitinib or salt may contain from about 1% to about 20% (w/w), from about 5% to about 20% (w/w), or from about 5% to about 10% (w/w) of one or more other crystalline polymorph of Gusacitinib and/or of Gusacitinib salt.

Depending on which other crystalline polymorphs a comparison is made, the crystalline polymorphs of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Gusacitinib referred to herein as being characterized by graphical data “as depicted in” or “as substantially depicted in” a Figure will thus be to include any crystal forms of Gusacitinib characterized with the graphical data having such small variations, as are well known to the skilled person, in comparison with the Figure.

As used herein, and unless stated otherwise, the term “anhydrous” in relation to crystalline forms of Gusacitinib, relates to a crystalline form of Gusacitinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, unless stated otherwise, the XRPD measurements are taken using copper Kα radiation wavelength 1.5418 Å. XRPD peaks reported herein are measured using Cuk α radiation, λ=1.5418 Å, typically at a temperature of 25±3° C. or at about 22-25° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

In one embodiment, the present invention provides crystalline Gusacitinib and crystalline Gusacitinib HCl salt.

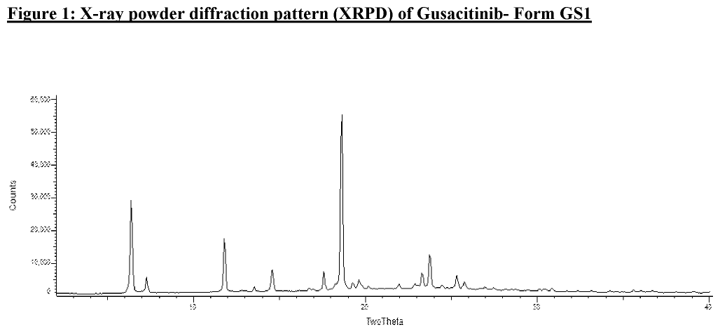

The present disclosure includes a crystalline polymorph of Gusacitinib-designated GS1. The crystalline Form GS1 of Gusacitinib may be characterized by data selected from one or more of the following: an X-ray powder diffraction pattern substantially as depicted in FIG. 1; an X-ray powder diffraction pattern having peaks at 6.4, 7.3, 11.8, 17.6 and 23.8 degrees 2-theta±0.2 degrees 2-theta; and combinations of these data.

Crystalline Form GS1 may be further characterized by an X-ray powder diffraction pattern having peaks at 6.4, 7.3, 11.8, 17.6 and 23.8 degrees 2-theta±0.2 degrees 2-theta, and also having any one, two, or three additional peaks selected from 14.6, 18.6 and 25.8 degrees 2-theta±0.2 degrees 2-theta.

In one embodiment of the present disclosure, crystalline Form GS1 of Gusacitinib is isolated.

In another embodiment crystalline Form GS1 may be a solvated form; in particular, DMF-water solvate.

Form GS1 according to any aspect or embodiment of the present disclosure may be polymorphically pure.

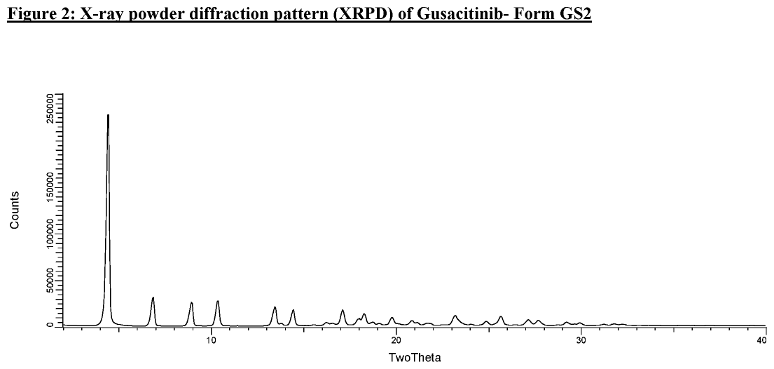

In another embodiment, the present disclosure includes a crystalline polymorph of Gusacitinib-designated GS2. The crystalline Form GS2 of Gusacitinib may be characterized by data selected from one or more of the following: an X-ray powder diffraction pattern substantially as depicted in FIG. 2; an X-ray powder diffraction pattern having peaks at 4.4, 6.8, 8.9, and 10.3 degrees 2-theta±0.2 degrees 2-theta, and also having any one, two, three, four or five additional peaks 13.4, 17.1, 18.3, 20.8 and 23.2 degrees 2-theta±0.2 degrees 2-theta; and combinations of these data.

Alternatively, Gusacitinib Form GS2 may be characterized by an X-ray powder diffraction pattern having peaks at 4.4, 6.8, 8.9, 10.3, 20.8 degrees 2-theta±0.2 degrees 2-theta. Gusacitinib Form GS2 may be alternatively characterised by an X-ray powder diffraction pattern having peaks at 4.4, 6.8, 8.9, 10.3, 20.8 degrees 2-theta±0.2 degrees 2-theta, and optionally having any one, two, three, or four additional peaks 13.4, 17.1, 18.3, and 23.2 degrees 2-theta±0.2 degrees 2-theta.

Crystalline Form GS2 of Gusacitinib may be further characterized by an X-ray powder diffraction pattern having peaks at 4.4, 6.8, 8.9, 10.3, 13.4, 17.1, 18.3, 20.8 and 23.2 degrees 2-theta±0.2 degrees 2-theta.

In one embodiment of the present disclosure, crystalline Form GS2 of Gusacitinib is isolated.

In another embodiment Crystalline Form GS2 of Gusacitinib may be anhydrous.

Form GS2 according to any aspect or embodiment of the present disclosure may be polymorphically pure.

Form GS2 of Gusacitinib is stable under all tested stress conditions (e.g., under strong grinding, pressure of 2 tons, high humidity (up to 100% RH for 7 days) and at high temperature (up to 100° C.).

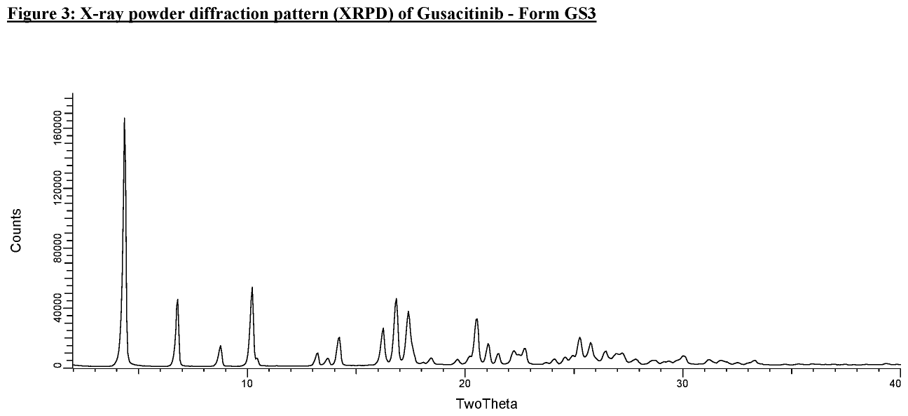

In another embodiment, the present disclosure provides a crystalline polymorph of Gusacitinib-designated GS3. The crystalline Form GS3 of Gusacitinib may be characterized by data selected from one or more of the following: an X-ray powder diffraction pattern substantially as depicted in FIG. 3; an X-ray powder diffraction pattern having peaks at 4.4, 6.8, 8.9 and 10.3 degrees 2-theta±0.2 degrees 2-theta, and also having any one, two, three, four or five additional peaks selected from 14.2, 16.8, 17.5, 21.1 and 25.2 degrees 2-theta±0.2 degrees 2-theta; and combinations of these data.

Alternatively, Gusacitinib Form GS3 may be characterized by an X-ray powder diffraction pattern having peaks at 4.4, 6.8, 8.9, 10.3, and 17.5 degrees 2-theta±0.2 degrees 2-theta, and optionally having any one, two, three, or four additional peaks 14.2, 16.8, 21.1 and 25.2 degrees 2-theta±0.2 degrees 2-theta.

Gusacitinib Form GS3 may be alternatively characterized by an X-ray powder diffraction pattern having peaks at 4.4, 6.8, 8.9, 10.3 and 13.1 degrees 2-theta±0.2 degrees 2-theta, and also having any one, two, three, four or five additional peaks selected from 14.2, 16.8, 17.5, 21.1 and 25.2 degrees 2-theta±0.2 degrees 2-theta; or an X-ray powder diffraction pattern having peaks at 4.4, 6.8, 8.9, 10.3 and 24.1 degrees 2-theta±0.2 degrees 2-theta, and also having any one, two, three, four or five additional peaks selected from 14.2, 16.8, 17.5, 21.1 and 25.2 degrees 2-theta±0.2 degrees 2-theta; or an X-ray powder diffraction pattern having peaks at 4.4, 6.8, 8.9, 10.3 and 28.5 degrees 2-theta±0.2 degrees 2-theta, and also having any one, two, three, four or five additional peaks selected from 14.2, 16.8, 17.5, 21.1 and 25.2 degrees 2-theta±0.2 degrees 2-theta.

Gusacitinib Form GS3 may be alternatively characterized by an X-ray powder diffraction pattern having peaks at 4.4, 6.8, 8.9, 10.3, and: any two or three peaks at 13.1, 24.1 or 28.5 (particularly the two peaks 13.1 and 24.1, or the two peaks at 13.1 and 28.5, or the two peaks at 24.1 and 28.5, or the three peaks at 13.1, 24.1 and 28.5) degrees 2-theta #0.2 degrees 2-theta, and additionally having any one, two, three, four or five additional peaks selected from 14.2, 16.8, 17.5, 21.1 and 25.2 degrees 2-theta±0.2 degrees 2-theta.

Alternatively, Gusacitinib Form GS3 may be characterized by an X-ray powder diffraction pattern having peaks at:
4.4, 6.8, 8.9, 10.3, 13.1, and 17.5 degrees 2-theta±0.2 degrees 2-theta, and optionally having any one, two, three, or four additional peaks 14.2, 16.8, 21.1 and 25.2 degrees 2-theta±0.2 degrees 2-theta (particularly, Form GS3 may be characterized by an XRPD pattern having peaks at 4.4, 6.8, 8.9, 10.3, 13.1, 14.2, 16.8, 17.5, 21.1, and 25.2); or
4.4, 6.8, 8.9, 10.3, 17.5, and 24.1 degrees 2-theta±0.2 degrees 2-theta, and optionally having any one, two, three, or four additional peaks 14.2, 16.8, 21.1 and 25.2 degrees 2-theta±0.2 degrees 2-theta (particularly, Form GS3 may be characterized by an XRPD pattern having peaks at 4.4, 6.8, 8.9, 10.3, 14.2, 16.8, 17.5, 21.1, 24.1 and 25.2 degrees 2-theta±0.2 degrees 2-theta); or
4.4, 6.8, 8.9, 10.3, 17.5, and 28.5 degrees 2-theta±0.2 degrees 2-theta, and optionally having any one, two, three, or four additional peaks 14.2, 16.8, 21.1 and 25.2 degrees 2-theta±0.2 degrees 2-theta (particularly, Form GS3 may be characterized by an XRPD pattern having peaks at 4.4, 6.8, 8.9, 10.3, 14.2, 16.8, 17.5, 21.1, 25.2, and 28.5 degrees 2-theta±0.2 degrees 2-theta) or
4.4, 6.8, 8.9, 10.3, 13.1, 17.5, and 24.1 degrees 2-theta±0.2 degrees 2-theta, and optionally having any one, two, three, or four additional peaks 14.2, 16.8, 21.1 and 25.2 degrees 2-theta #0.2 degrees 2-theta (particularly, Form GS3 may be characterized by an XRPD pattern having peaks at 4.4, 6.8, 8.9, 10.3, 13.1, 14.2, 16.8, 17.5, 21.1, 24.1, and 25.2 degrees 2-theta±0.2 degrees 2-theta); or
4.4, 6.8, 8.9, 10.3, 13.1, 17.5, and 28.5 degrees 2-theta±0.2 degrees 2-theta, and optionally having any one, two, three, or four additional peaks 14.2, 16.8, 21.1 and 25.2 degrees 2-theta±0.2 degrees 2-theta (particularly, Form GS3 may be characterized by an XRPD pattern having peaks at 4.4, 6.8, 8.9, 10.3, 13.1, 14.2, 16.8, 17.5, 21.1, 25.2, and 28.5 degrees 2-theta±0.2 degrees 2-theta); or
4.4, 6.8, 8.9, 10.3, 17.5, 24.1, and 28.5 degrees 2-theta±0.2 degrees 2-theta, and optionally having any one, two, three, or four additional peaks 14.2, 16.8, 21.1 and 25.2 degrees 2-theta±0.2 degrees 2-theta (particularly, Form GS3 may be characterized by an XRPD pattern having peaks at 4.4, 6.8, 8.9, 10.3, 14.2, 16.8, 17.5, 21.1, 24.1, 25.2, and 28.5 degrees 2-theta±0.2 degrees 2-theta); or
4.4, 6.8, 8.9, 10.3, 13.1, 17.5, 24.1, and 28.5 degrees 2-theta±0.2 degrees 2-theta, and optionally having any one, two, three, or four additional peaks 14.2, 16.8, 21.1 and 25.2 degrees 2-theta±0.2 degrees 2-theta (particularly, Form GS3 may be characterized by an XRPD pattern having peaks at 4.4, 6.8, 8.9, 10.3, 13.1, 14.2, 16.8, 17.5, 21.1, 24.1, 25.2, and 28.5 degrees 2-theta±0.2 degrees 2-theta).

Crystalline Form GS3 of Gusacitinib may be further characterized by an X-ray powder diffraction pattern having peaks at 4.4, 6.8, 8.9, 10.3, 14.2, 16.8, 17.5, 21.1 and 25.2 degrees 2-theta±0.2 degrees 2-theta.

In any embodiment or aspect of the present disclosure, crystalline Form GS3 of Gusacitinib is isolated. In any embodiment or aspect of the present disclosure, crystalline Form GS3 of Gusacitinib is anhydrous.

In one embodiment of the present disclosure, crystalline Form GS3 of Gusacitinib is isolated.

Crystalline Form GS3 may be anhydrous.

Form GS3 according to any aspect or embodiment of the present disclosure may be polymorphically pure.

Form GS3 of Gusacitinib is stable under all tested stress conditions (e.g., under strong grinding, pressure of 2 tons, high humidity (up to 100% RH for 7 days) and at high temperature (up to 100° C.).

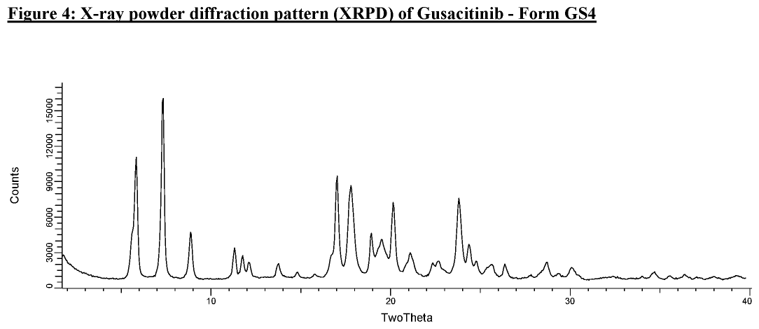

In another embodiment, the present disclosure includes a crystalline polymorph of Gusacitinib-designated GS4. The crystalline Form GS4 of Gusacitinib may be characterized by data selected from one or more of the following: an X-ray powder diffraction pattern substantially as depicted in FIG. 4; an X-ray powder diffraction pattern having peaks at 5.9, 7.4, 11.3, 20.2, 24.4 degrees 2-theta±0.2 degrees 2-theta, and also having any one, two, three, four or five additional peaks 11.8, 12.1, 17.0, 17.8 and 23.8 degrees 2-theta±0.2 degrees 2-theta; and combinations of these data.

Alternatively Form GS4 of Gusacitinib may be characterized an X-ray powder diffraction pattern having peaks at 5.9, 7.4, 11.3, 11.8, 20.2, 24.4 degrees 2-theta±0.2 degrees 2-theta, and also having any one, two, three, or four additional peaks 12.1, 17.0, 17.8 and 23.8 degrees 2-theta±0.2 degrees 2-theta; and combinations of these data. Form GS4 of Gusacitinib may optionally be characterized an X-ray powder diffraction pattern having peaks at 5.9, 7.4, 11.3, 12.1, 20.2, 24.4 degrees 2-theta±0.2 degrees 2-theta, and also having any one, two, three, or four additional peaks 11.8, 17.0, 17.8 and 23.8 degrees 2-theta±0.2 degrees 2-theta. Alternatively Form GS4 of Gusacitinib may optionally be characterized an X-ray powder diffraction pattern having peaks at 5.9, 7.4, 11.3, 11.8, 12.1, 20.2, 24.4 degrees 2-theta±0.2 degrees 2-theta, and also having any one, two or three additional peaks 17.0, 17.8 and 23.8 degrees 2-theta±0.2 degrees 2-theta.

Crystalline Form GS4 of Gusacitinib may be further characterized by an X-ray powder diffraction pattern having peaks at 5.9, 7.4, 11.3, 11.8, 12.1, 17.0, 17.8, 20.2, 23.8 and 24.4 degrees 2-theta±0.2 degrees 2-theta.

In one embodiment of the present disclosure, crystalline Form GS4 of Guanidine is isolated.

In another embodiment Crystalline Form GS4 of Gusacitinib may be anhydrous.

Form GS4 according to any aspect or embodiment of the present disclosure may be polymorphically pure.

Form GS4 of Gusacitinib is stable under all tested stress conditions (e.g., under strong grinding, pressure of 2 tons, high humidity (up to 80% RH for 7 days) and at high temperature (up to 100° C.).

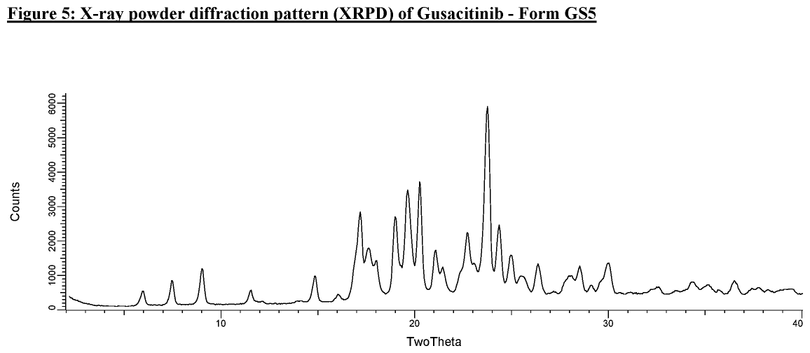

In a further embodiment, the present disclosure provides a crystalline polymorph of Gusacitinib-designated GS5. The crystalline Form GS5 of Gusacitinib may be characterized by data selected from one or more of the following: an X-ray powder diffraction pattern substantially as depicted in FIG. 5; an X-ray powder diffraction pattern having peaks at 17.4, 18.4, 20.9, 26.2 and 29.8 degrees 2-theta±0.2 degrees 2-theta, and also having any one, two, three, or four additional peaks selected from 8.9, 14.7, 20.1 and 23.6 degrees 2-theta±0.2 degrees 2-theta; and combinations of these data.

Alternatively, Form GS5 of Gusacitinib may be characterized by an X-ray powder diffraction pattern having a peak at 17.4, 18.8 20.9, 26.2 and 29.8 degrees 2-theta±0.2 degrees 2-theta, and also having any one, two, three, or four additional peaks selected from 8.9, 14.7, 20.1 and 23.6 degrees 2-theta±0.2 degrees 2-theta; and combinations of these data.

Alternatively Form GS5 of Gusacitinib may be characterized by an X-ray powder diffraction pattern having peaks at 17.4, 18.4, 20.1, 20.9, 26.2 and 29.8 degrees 2-theta±0.2 degrees 2-theta, and also having any one, two or three additional peaks selected from 8.9, 14.7 and 23.6 degrees 2-theta±0.2 degrees 2-theta; and combinations of these data. Further alternatively, Form GS5 of Gusacitinib may be characterized by an X-ray powder diffraction pattern having a peak at 17.4, 18.8, 20.1, 20.9, 26.2 and 29.8 degrees 2-theta±0.2 degrees 2-theta, and also having any one, two, three, or four additional peaks selected from 8.9, 14.7 and 23.6 degrees 2-theta±0.2 degrees 2-theta; and combinations of these data.

Crystalline Form GS5 of Gusacitinib may be further characterized by an X-ray powder diffraction pattern having peaks at 8.9, 14.7, 17.4, 18.4, 20.1, 20.9, 23.6, 26.2 and 29.8 degrees 2-theta±0.2 degrees 2-theta.

In one embodiment of the present disclosure, crystalline Form GS5 of Gusacitinib is isolated.

Crystalline Form GS5 may be a DMSO solvate.

Form GS5 according to any aspect or embodiment of the present disclosure may be polymorphically pure.

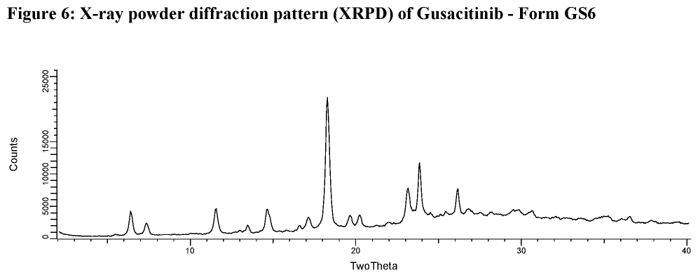

In another embodiment, the present disclosure includes a crystalline polymorph of Gusacitinib-designated GS6. The crystalline Form GS6 of Gusacitinib may be characterized by data selected from one or more of the following: an X-ray powder diffraction pattern substantially as depicted in FIG. 6; an X-ray powder diffraction pattern having peaks at 11.6, 14.7, 18.3, 19.6 and 23.9 degrees 2-theta±0.2 degrees 2-theta; and combinations of these data.

Crystalline Form GS6 of Gusacitinib may be characterized by an X-ray powder diffraction pattern having peaks at 11.6, 14.7, 18.3, 19.6 and 23.9 degrees 2-theta±0.2 degrees 2-theta, and also having any one, two, three, or four additional peaks selected from 7.4, 13.5, 20.2 and 26.2 degrees 2-theta±0.2 degrees 2-theta.

Crystalline Form GS6 of Gusacitinib may be further characterized by an X-ray powder diffraction pattern having peaks at 7.4, 11.6, 13.5, 14.7, 18.3, 19.6, 20.2, 23.9 and 26.2 degrees 2-theta±0.2 degrees 2-theta.

In one embodiment of the present disclosure, crystalline Form GS6 of Gusacitinib is isolated.

In another embodiment, Crystalline Form GS6 of Gusacitinib may be hydrate, preferably containing water in amount of: about 13 to about 25 wt %; or about 16 to about 22 wt %. More preferably Form GS6 of Gusacitinib as described in any aspect or embodiment of the disclosure may be a pentahydrate

Form GS6 according to any aspect or embodiment of the present disclosure may be polymorphically pure.

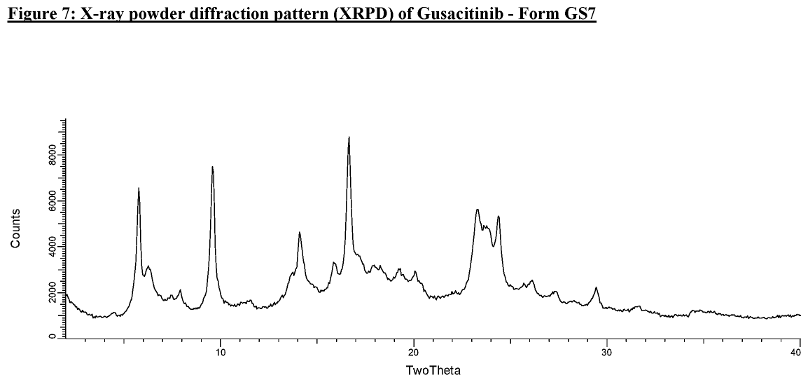

In a further embodiment, the present disclosure provides a crystalline polymorph of Gusacitinib-designated GS7. The crystalline Form GS7 of Gusacitinib may be characterized by data selected from one or more of the following: an X-ray powder diffraction pattern substantially as depicted in FIG. 7; an X-ray powder diffraction pattern having peaks at 6.3, 9.6, 16.7, 23.3 and 26.1 degrees 2-theta±0.2 degrees 2-theta; and combinations of these data.

Crystalline Form GS7 may be characterized by an X-ray powder diffraction pattern having peaks at 6.3, 9.6, 16.7, 23.3 and 26.1 degrees 2-theta±0.2 degrees 2-theta, and also having any one, two, or three additional peaks selected from 5.8, 20.1 and 29.5 degrees 2-theta±0.2 degrees 2-theta.

Crystalline Form GS7 of Gusacitinib may be further characterized by an X-ray powder diffraction pattern having peaks at 5.8, 6.3, 9.6, 16.7, 20.1, 23.3, 26.1 and 29.5 degrees 2-theta±0.2 degrees 2-theta.

In one embodiment of the present disclosure, crystalline Form GS7 of Gusacitinib is isolated.

Form GS7 of Gusacitinib as described in any aspect or embodiment of the disclosure may contain water in an amount of: about 0.5 to about 3.0 wt %; about 0.8 to about 3.8 wt %, or about 1.0 to about 2.5 wt %.

Form GS7 of Gusacitinib as described in any aspect or embodiment of the disclosure may be a hydrate, preferably containing water in amount of: about 0.5 to about 3.0 wt %; about 0.8 to about 3.8 wt %, or about 1.0 to about 2.5 wt %. More preferably Form GS7 of Gusacitinib as described in any aspect or embodiment of the disclosure may be a hemihydrate.

Form GS7 of Gusacitinib may be a hydrate; more preferably a hemihydrate.

Form GS7 according to any aspect or embodiment of the present disclosure may be polymorphically pure.

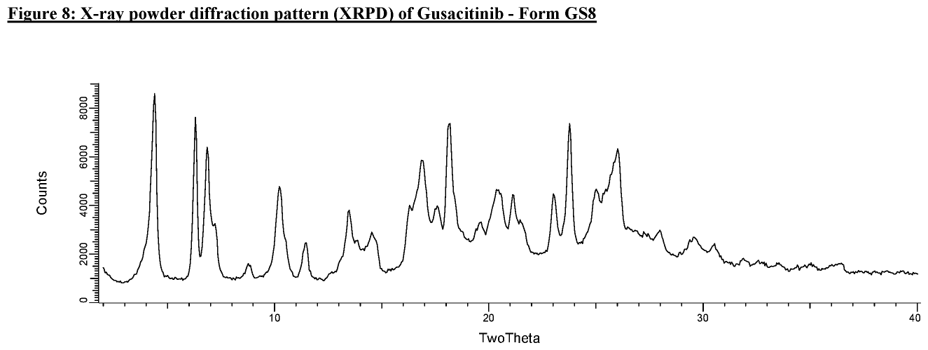

In a further embodiment, the present disclosure provides a crystalline polymorph of Gusacitinib-designated GS8. The crystalline Form GS8 of Gusacitinib may be characterized by data selected from one or more of the following: an X-ray powder diffraction pattern substantially as depicted in FIG. 8; an X-ray powder diffraction pattern having peaks at 6.9, 10.2, 23.0, 25.8 and 27.9 degrees 2-theta±0.2 degrees 2-theta; and combinations of these data.

Crystalline From GS8 may be characterized by an X-ray powder diffraction pattern having peaks at 6.9, 10.2, 23.0, 25.8 and 27.9 degrees 2-theta±0.2 degrees 2-theta, and also having any one, two or three, additional peaks selected from 4.4, 16.9 and 21.1 degrees 2-theta±0.2 degrees 2-theta.

Crystalline Form GS8 of Gusacitinib may be further characterized by an X-ray powder diffraction pattern having peaks at 4.4, 6.9, 10.2, 16.9, 21.1, 23.0, 25.8 and 27.9 degrees 2-theta±0.2 degrees 2-theta.

In one embodiment of the present disclosure, crystalline Form GS8 of Gusacitinib is isolated.

Crystalline Form GS8 of Gusacitinib as described in any aspect or embodiment of the disclosure may contain water in an amount of: about 1.8 to about 5.8 wt %; about 2.2 to about 5.5 wt %, or about 2.5 to about 5.0 wt %.

Crystalline Form GS8 as described in any aspect or embodiment of the disclosure may be hydrate, preferably containing water in an amount of: about 1.8 to about 5.8 wt %; about 2.2 to about 5.5 wt %, or about 2.5 to about 5.0 wt %. More preferably crystalline Form GS8 as described in any aspect or embodiment of the disclosure is a monohydrate.

Crystalline Form GS8 may be hydrate and more preferably a monohydrate.

Form GS8 according to any aspect or embodiment of the present disclosure may be polymorphically pure.

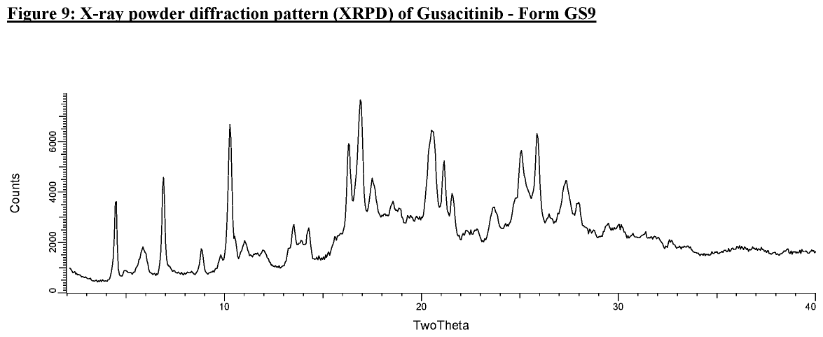

In a further embodiment, the present disclosure provides a crystalline polymorph of Gusacitinib-designated GS9. The crystalline Form GS9 of Gusacitinib may be characterized by data selected from one or more of the following: an X-ray powder diffraction pattern substantially as depicted in FIG. 9; an X-ray powder diffraction pattern having peaks at 5.9, 8.8, 10.3, 16.9 and 25.1 degrees 2-theta±0.2 degrees 2-theta; and combinations of these data.

Crystalline Form GS9 of Gusacitinib may be further characterized by an X-ray powder diffraction pattern having peaks at 5.9, 8.8, 10.3, 16.9 and 25.1 degrees 2-theta±0.2 degrees 2-theta, and also having any one, two, three, or four additional peaks selected from 17.5, 21.2, 23.7 and 28.0 degrees 2-theta±0.2 degrees 2-theta.

Crystalline Form GS9 of Gusacitinib may be further characterized by an X-ray powder diffraction pattern having peaks at 5.9, 8.8, 10.3, 16.9, 17.5, 21.2, 23.7, 25.1 and 28.0 degrees 2-theta±0.2 degrees 2-theta.

In one embodiment of the present disclosure, crystalline Form GS9 of Gusacitinib is isolated.

Form GS9 according to any aspect or embodiment of the present disclosure may be polymorphically pure.

In another embodiment, the present invention discloses a crystalline Gusacitinib HCl salt.

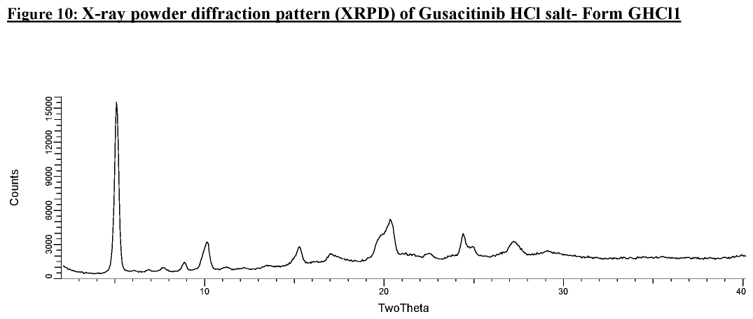

In a further embodiment, the present disclosure discloses a crystalline polymorph of Gusacitinib HCl salt; designated GHC11. The crystalline Form GHC11 of Gusacitinib HCl salt may be characterized by data selected from one or more of the following: an X-ray powder diffraction pattern substantially as depicted in FIG. 10; an X-ray powder diffraction pattern having peaks at 5.1, 8.9, 10.1, 15.3 and 27.2 degrees 2-theta±0.2 degrees 2-theta; and combinations of these data.

Crystalline Form GHC11 of Gusacitinib HCl salt may be further characterized by an X-ray powder diffraction pattern having peaks at 5.1, 8.9, 10.1, 15.3 and 27.2 degrees 2-theta #0.2 degrees 2-theta, and also having any one, two, three, or four additional peaks selected from 17.5, 21.2, 23.7 and 28.0 degrees 2-theta±0.2 degrees 2-theta. Alternatively, Form GHC11 of Gusacitinib HCl salt may be characterized by an X-ray powder diffraction pattern having peaks at 5.1, 8.9, 10.1, 15.3 and 27.2 degrees 2-theta±0.2 degrees 2-theta, and also having any one, two or three, additional peaks selected from 17.1, 20.4 and 24.4 degrees 2-theta±0.2 degrees 2-theta.

In a further embodiment, crystalline Form GHC11 of Gusacitinib HCl salt may be characterized by an X-ray powder diffraction pattern having peaks at 5.1, 8.9, 10.1, 15.3, 17.5, 21.2, 23.7, 27.2 and 28.0 degrees 2-theta±0.2 degrees 2-theta. Alternatively, Form GHC11 of Gusacitinib HCl salt may be characterized by an X-ray powder diffraction pattern having peaks at 5.1, 8.9, 10.1, 15.3, 17.1, 20.4, 24.4, and 27.2 degrees 2-theta±0.2 degrees 2-theta.

Crystalline Form GHC11 of Gusacitinib HCl salt as described in any aspect or embodiment of the disclosure may contain water in an amount of: about 6.5 to about 13.5 wt %; about 7.5 to about 12.5 wt %, or about 8.0 to about 12.0 wt %. Crystalline Form GHC11 of Gusacitinib HCl salt as described in any aspect or embodiment of the disclosure may be a hydrate, preferably containing water in an amount of: about 6.5 to about 13.5 wt %; about 7.5 to about 12.5 wt %, or about 8.0 to about 12.0 wt %. More preferably, crystalline form GHC11 according to any aspect or embodiment of the disclosure is a trihydrate.

In another embodiment, crystalline Form GHC11 of Gusacitinib HCl salt is a hydrate; preferably trihydrate.

Form GHC11 according to any aspect or embodiment of the present disclosure may be polymorphically pure.

Form GHC11 of Gusacitinib HCl salt is stable under all tested stress conditions (e.g., under strong grinding, pressure of 2 tons, high humidity (up to 100% RH for 7 days) and at high temperature (up to 100° C.).

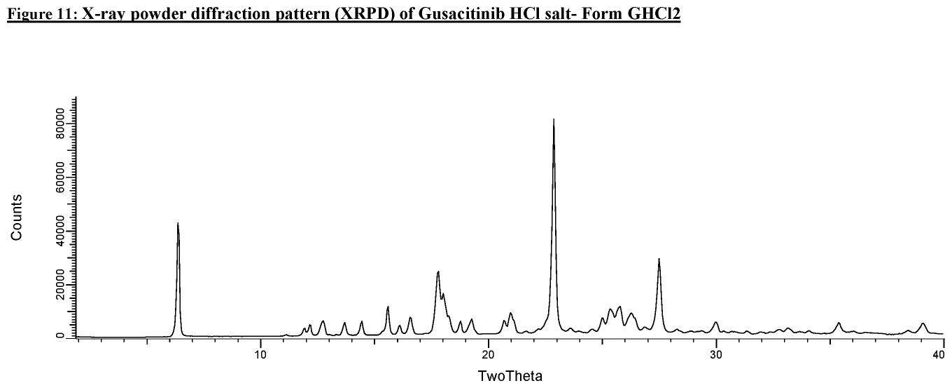

In another embodiment, the present disclosure discloses a crystalline polymorph of Gusacitinib HCl salt; designated GHC12. The crystalline Form GHC12 of Gusacitinib HCl salt may be characterized by data selected from one or more of the following: an X-ray powder diffraction pattern substantially as depicted in FIG. 11; an X-ray powder diffraction pattern having peaks at 6.4, 13.7, 15.6, 22.9 and 27.5 degrees 2-theta±0.2 degrees 2-theta; and combinations of these data.

Crystalline Form GHC12 of Gusacitinib HCl salt may be further characterized by an X-ray powder diffraction pattern having peaks at 6.4, 13.7, 15.6, 22.9 and 27.5 degrees 2-theta±0.2 degrees 2-theta, and also having any one, two, three, or four additional peaks selected from 12.7, 14.4, 16.6 and 17.8 degrees 2-theta±0.2 degrees 2-theta.

In a further embodiment, crystalline Form GHC12 of Gusacitinib HCl salt may be characterized by an X-ray powder diffraction pattern having peaks at 6.4, 12.7, 13.7, 14.4, 15.6, 16.6, 17.8, 22.9 and 27.5 degrees 2-theta±0.2 degrees 2-theta.

Crystalline Form GHC12 of Gusacitinib HCl salt may be anhydrous.

The above crystalline polymorphs of Gusacitinib/Gusacitinib salts can be used to prepare other crystalline polymorphs of Gusacitinib, salts of Gusacitinib and solid state forms thereof.

Form GHC12 according to any aspect or embodiment of the present disclosure may be polymorphically pure.

Form GHC12 of Gusacitinib HCl salt is stable under all tested stress conditions (e.g., under strong grinding, pressure of 2 tons, high humidity (up to 100% RH for 7 days) and at high temperature (up to 100° C.).

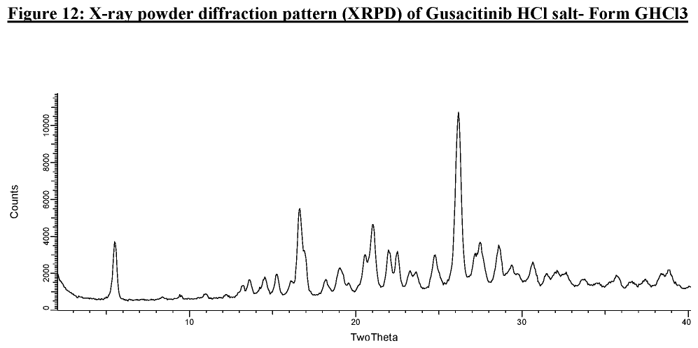

In another embodiment, the present disclosure discloses a crystalline polymorph of Gusacitinib HCl salt; designated GHC13. The crystalline Form GHC13 of Gusacitinib HCl salt may be characterized by data selected from one or more of the following: an X-ray powder diffraction pattern substantially as depicted in FIG. 12; an X-ray powder diffraction pattern having peaks at 5.5, 16.7, 21.0, 22.0 and 26.2 degrees 2-theta±0.2 degrees 2-theta; and combinations of these data.

Crystalline Form GHC13 of Gusacitinib HCl salt may be further characterized by an X-ray powder diffraction pattern having peaks at 5.5, 16.7, 21.0, 22.0 and 26.2 degrees 2-theta±0.2 degrees 2-theta, and also having any one, two, three, or four additional peaks selected from 14.5, 19.0, 24.8 and 28.6 degrees 2-theta±0.2 degrees 2-theta.

In a further embodiment, crystalline Form GHC13 of Gusacitinib HCl salt may be characterized by an X-ray powder diffraction pattern having peaks at 5.5, 14.5, 16.7, 19.0, 21.0, 22.0, 24.8, 26.2 and 28.6 degrees 2-theta±0.2 degrees 2-theta.

Crystalline Form GHC13 of Gusacitinib HCl salt as described in any aspect or embodiment of the disclosure may contain water in an amount of: about 6.5 to about 13.5 wt %; about 7.5 to about 12.5 wt %, or about 8.0 to about 12.0 wt %. Crystalline Form GHC13 of Gusacitinib HCl salt as described in any aspect or embodiment of the disclosure may be a hydrate, preferably containing water in an amount of: about 6.5 to about 13.5 wt %; about 7.5 to about 12.5 wt %, or about 8.0 to about 12.0 wt %. More preferably, crystalline form GHC13 according to any aspect or embodiment of the disclosure is a trihydrate.

In another embodiment, crystalline Form GHC13 of Gusacitinib HCl salt is a hydrated form; preferably a trihydrate form.

Form GHC13 according to any aspect or embodiment of the present disclosure may be polymorphically pure.

Form GHC13 of Gusacitinib HCl salt is stable under tested stress conditions (e.g., under strong grinding; pressure of 2 tons and high humidity (up to 60% RH for 7 days).

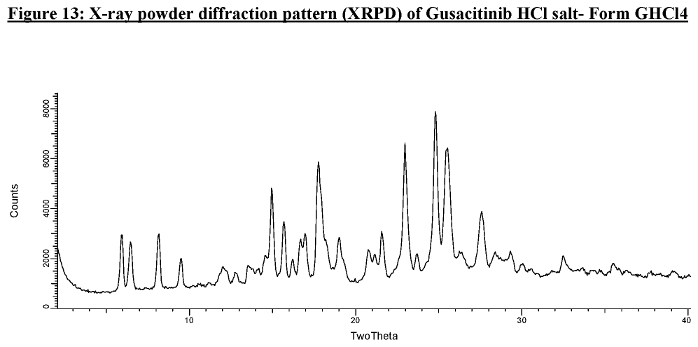

In a further embodiment, the present disclosure discloses a crystalline polymorph of Gusacitinib HCl salt; designated GHC14. The crystalline Form GHC14 of Gusacitinib HCl salt may be characterized by data selected from one or more of the following: an X-ray powder diffraction pattern substantially as depicted in FIG. 13; an X-ray powder diffraction pattern having peaks at 5.8, 8.0, 14.9, 21.5 and 25.4 degrees 2-theta±0.2 degrees 2-theta; and combinations of these data.

Crystalline Form GHC14 of Gusacitinib HCl salt may be further characterized by an X-ray powder diffraction pattern having peaks at 5.8, 8.0, 14.9, 21.5 and 25.4 degrees 2-theta±0.2 degrees 2-theta, and also having anyone, two, three, four, or five additional peaks selected from 9.4, 12.7, 15.6, 17.8 and 22.9 degrees 2-theta±0.2 degrees 2-theta.

In a further embodiment, crystalline Form GHC14 of Gusacitinib HCl salt may be characterized by an X-ray powder diffraction pattern having peaks at 5.8, 8.0, 9.4, 12.7, 14.9, 15.6, 17.8, 21.5, 22.9 and 25.4 degrees 2-theta±0.2 degrees 2-theta.

In another embodiment, crystalline Form GHC14 of Gusacitinib HCl salt is an anhydrous form. Form GHC14 according to any aspect or embodiment of the present disclosure may be polymorphically pure.

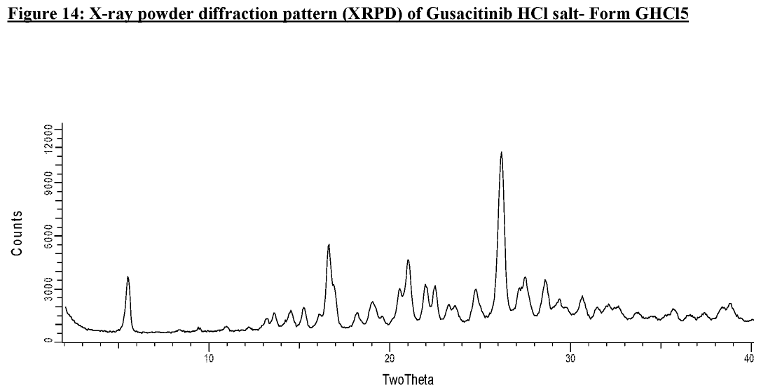

In another embodiment, the present disclosure discloses a crystalline polymorph of Gusacitinib HCl salt; designated GHC15. The crystalline Form GHC15 of Gusacitinib HCl salt may be characterized by data selected from one or more of the following: an X-ray powder diffraction pattern substantially as depicted in FIG. 14; an X-ray powder diffraction pattern having peaks at 5.2, 8.8, 16.6, 23.9 and 25.5 degrees 2-theta±0.2 degrees 2-theta; and combinations of these data.

Crystalline Form GHC15 of Gusacitinib HCl salt may be further characterized by an X-ray powder diffraction pattern having peaks at 5.2, 8.8, 16.6, 23.9 and 25.5 degrees 2-theta±0.2 degrees 2-theta, and also having anyone, two, or three additional peaks selected from 17.7, 20.1 and 21.7 degrees 2-theta±0.2 degrees 2-theta.

In a further embodiment, crystalline Form GHC15 of Gusacitinib HCl salt may be characterized by an X-ray powder diffraction pattern having peaks at 5.2, 8.8, 16.6, 17.7, 20.1, 21.7, 23.9 and 25.5 degrees 2-theta±0.2 degrees 2-theta.

In another embodiment, crystalline Form GHC15 of Gusacitinib HCl salt is a solvated form; preferably 1,4 dioxane solvate.

Form GHC15 according to any aspect or embodiment of the present disclosure may be polymorphically pure.

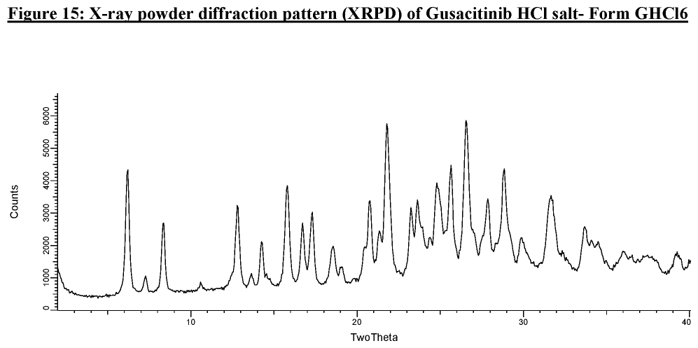

In another embodiment, the present disclosure discloses a crystalline polymorph of Gusacitinib HCl salt; designated GHC16. The crystalline Form GHC16 of Gusacitinib HCl salt may be characterized by data selected from one or more of the following: an X-ray powder diffraction pattern substantially as depicted in FIG. 15; an X-ray powder diffraction pattern having peaks at 6.2, 12.8, 17.3, 20.8 and 26.6 degrees 2-theta±0.2 degrees 2-theta; and combinations of these data.

Crystalline Form GHC16 of Gusacitinib HCl salt may be further characterized by an X-ray powder diffraction pattern having peaks at 6.2, 12.8, 17.3, 20.8 and 26.6 degrees 2-theta±0.2 degrees 2-theta, and also having any one, two, three, or four additional peaks selected from 14.3, 21.8, 25.7 and 31.7 degrees 2-theta±0.2 degrees 2-theta.

In a further embodiment, crystalline Form GHC16 of Gusacitinib HCl salt may be characterized by an X-ray powder diffraction pattern having peaks at 6.2, 12.8, 14.3, 17.3, 20.8, 21.8, 25.7, 26.6 and 31.7 degrees 2-theta±0.2 degrees 2-theta.

In another embodiment, crystalline Form GHC16 of Gusacitinib HCl salt may be a solvated form, preferably a mixture of water and 1,3 dioxolane solvate.

Form GHC16 according to any aspect or embodiment of the present disclosure may be polymorphically pure.

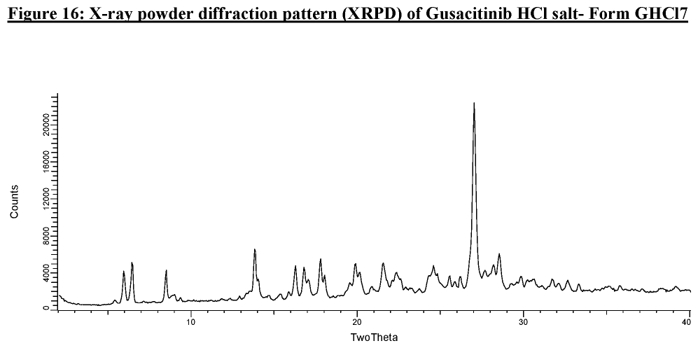

In a further embodiment, the present disclosure discloses a crystalline polymorph of Gusacitinib HCl salt; designated GHC17. The crystalline Form GHC17 of Gusacitinib HCl salt may be characterized by data selected from one or more of the following: an X-ray powder diffraction pattern substantially as depicted in FIG. 16; an X-ray powder diffraction pattern having peaks at 6.0, 8.5, 13.9, 16.3 and 27.0 degrees 2-theta±0.2 degrees 2-theta; and combinations of these data.

Crystalline Form GHC17 of Gusacitinib HCl salt may be further characterized by an X-ray powder diffraction pattern having peaks at 6.0, 8.5, 13.9, 16.3 and 27.0 degrees 2-theta±0.2 degrees 2-theta, and also having any one, two, three, or four additional peaks selected from 17.8, 21.6, 28.6 and 33.3 degrees 2-theta±0.2 degrees 2-theta.

In a further embodiment, crystalline Form GHC17 of Gusacitinib HCl salt may be characterized by an X-ray powder diffraction pattern having peaks at 6.0, 8.5, 13.9, 16.3, 17.8, 21.6, 27.0, 28.6 and 33.3 degrees 2-theta±0.2 degrees 2-theta.

In another embodiment, crystalline Form GHC17 of Gusacitinib HCl salt is a solvated form; preferably 1,2-dichloroethane solvate.

Form GHC17 according to any aspect or embodiment of the present disclosure may be polymorphically pure.

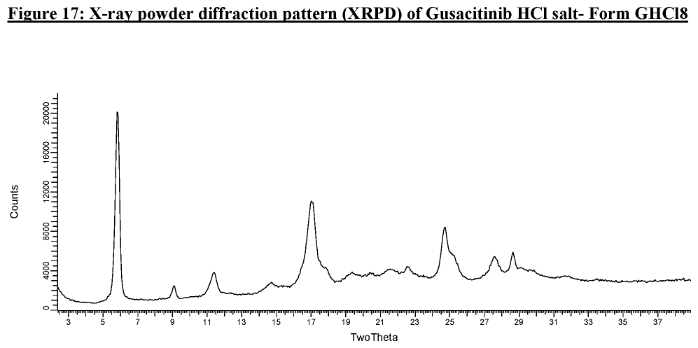

The present disclosure discloses a crystalline polymorph of Gusacitinib HCl salt; designated GHC18. The crystalline Form GHC18 of Gusacitinib HCl salt may be characterized by data selected from one or more of the following: an X-ray powder diffraction pattern substantially as depicted in FIG. 17; an X-ray powder diffraction pattern having peaks at 5.8, 11.4, 17.1, 24.6 and 27.5 degrees 2-theta±0.2 degrees 2-theta; and combinations of these data.

Crystalline Form GHC18 of Gusacitinib HCl salt may be further characterized by an X-ray powder diffraction pattern having peaks at 5.8, 11.4, 17.1, 24.6 and 27.5 degrees 2-theta±0.2 degrees 2-theta, and also having any one or two, additional peaks selected from 9.0 and 28.6 degrees 2-theta±0.2 degrees 2-theta.

In a further embodiment, crystalline Form GHC18 of Gusacitinib HCl salt may be characterized by an X-ray powder diffraction pattern having peaks at 5.8, 9.0, 11.4, 17.1, 24.6, 27.5 and 28.6 degrees 2-theta±0.2 degrees 2-theta.

Crystalline Form GHC18 of Gusacitinib HCl salt as described in any aspect or embodiment of the disclosure may contain water in an amount of: about 5.0 to about 10.0 wt %; about 5.5 to about 9.5 wt %, or about 6.0 to about 9.0 wt %. Crystalline Form GHC18 of Gusacitinib HCl salt as described in any aspect or embodiment of the disclosure may be a hydrate, preferably containing water in an amount of: about 5.0 to about 10.0 wt %; about 5.5 to about 9.5 wt %, or about 6.0 to about 9.0 wt %. More preferably, crystalline form GHC18 according to any aspect or embodiment of the disclosure is a dihydrate.

In another embodiment, crystalline Form GHC18 of Gusacitinib HCl salt is a hydrated form; preferably a dihydrate form.

Form GHC18 according to any aspect or embodiment of the present disclosure may be polymorphically pure.

Form GHC18 of Gusacitinib HCl salt is stable under all tested stress conditions (e.g., under strong grinding, pressure of 2 tons, high humidity (up to 100% RH for 7 days) and at high temperature (up to 100° C.).

The present disclosure provides crystalline polymorphs of Gusacitinib/Gusacitinib salts for use in the preparation of pharmaceutical compositions.

The present disclosure also encompasses the use of crystalline polymorphs of Gusacitinib of the present disclosure for the preparation of pharmaceutical compositions of crystalline polymorphs of Gusacitinib.

The present disclosure includes processes for preparing the above-mentioned pharmaceutical compositions. The processes include combining any one or a combination of the crystalline polymorphs of Gusacitinib of the present disclosure with at least one pharmaceutically acceptable excipient.

Pharmaceutical combinations or formulations of the present disclosure contain any one or a combination of the solid state forms of Gusacitinib of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Gusacitinib, and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Gusacitinib can be administered. Gusacitinib may be formulated for administration to a mammal, in embodiments to a human, by injection. Gusacitinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Gusacitinib, and the pharmaceutical compositions and/or formulations of Gusacitinib of the present disclosure, can be used as medicaments.

The present disclosure also provides methods of treating dermatitis by administering a therapeutically effective amount of any one or a combination of the crystalline polymorphs of Gusacitinib of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer:

Bruker D8 Advance; CuKα radiation (λ=1.5418 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring.

All X-Ray Powder Diffraction peak values are calibrated with regard to standard silicon spiking in the sample.

### EXAMPLES

### Preparation of Starting Materials

Gusacitinib can be prepared according to methods known from the literature, for example U.S. Pat. No. 8,729,079.

A pH 1.2 buffer may be prepared according any suitable method, particularly in accordance with the US Pharmacopoeia. A suitable buffer can be prepared using aqueous hydrochloric acid and potassium chloride solution:

The following Solutions 1 and 2 were prepared:
Solution 1: 8.5 ml conc. HCl+491.5 ml water
Solution 2: 745 g potassium chloride, made up to 500 ml with water.

Then, 425 ml of Solution 1 and 250 ml of Solution 2 were combined. Water was added to the combined solutions to make up to a total volume of 1000 ml. This solution can be used as the pH 1.2 buffer in the examples.

### Example 1: Preparation of Gusacitinib-Form GS1

Gusacitinib (0.05 g) was dissolved in N,N-dimethylformamide (DMF, 12 ml) at 25-30° C. The solution was filtered, water (10 ml) was added to the clear solution under stirring at 25-30° C. The reaction mixture was stirred for 1 hours and filtered under vacuum for about 15-20 minutes. The obtained solid was dried under vacuum at 25-30° C. for about 4 hours. The solid was analyzed by XRD and designated as Form GS1 of Gusacitinib; as shown in FIG. 1.

### Example 2: Preparation of Gusacitinib-Form GS2

Gusacitinib (1 g) was dissolved in a mixture of dichloromethane:methanol (1:1; 250 ml) at 25-30° C. The solution was filtered and clear solution was subjected to distillation under reduced pressure at 60° C. for about 30-45 minutes. The obtained solid was isolated, analyzed by XRD and designated as Form GS2 of Gusacitinib; as shown in FIG. 2.

### Example 3: Preparation of Gusacitinib-Form GS3

Gusacitinib (0.05 g) was dissolved in N-methyl pyrrolidone (NMP; 5 ml) at 25-30° C. The solution was filtered and in clear solution; water (10 ml) was added under stirring at 25-30° C. The reaction mixture was stirred for 1 hour and filtered under vacuum for 15-20 minutes. The obtained solid was analyzed by XRD and designated as Form GS3 of Gusacitinib; as shown in FIG. 3.

### Example 4: Preparation of Gusacitinib-Form GS4

Gusacitinib (0.6 g) was dissolved in N, N, dimethylformamide (DMF; 5 ml) at 60° C. The clear solution was added to a precooled (about 0° C.) of cyclopentyl methyl ether (5 ml). The obtained suspension maintained under stirring at 0° C. for 1 hour and filtered at 25° C. The filtered solid was dried under vacuum at 60° C. for 2 hours. The obtained solid was analyzed by XRD and designated as Gusacitinib Form GS4; as shown in FIG. 4.

### Example 5: Preparation of Gusacitinib-Form GS5

Gusacitinib (GS1, 100 mg) was taken in a test-tube and dissolved in dimethyl sulfoxide (DMSO, 0.8 ml) at 60° C. To the clear solution, methyl tert-butyl ether (MTBE, 6 ml) was added at 50° C. and maintained for 10-15 minutes at the same temperature. The reaction mixture was cooled to 25° C. and after 10-15 minutes the solution was filtered and suck dried under vacuum for 10-15 minutes. The solid was analyzed by XRD and designated as Form GS5 of Gusacitinib; as shown in FIG. 5.

### Example 6: Preparation of Gusacitinib-Form GS6

Gusacitinib (Form GS5, 0.1 g) and water (5 mL) were stirred at 25° C. The obtained slurry was stirred at 25° C. for 24 hours. The reaction mixture was filtered and dried under vacuum at 25-30° C. for 20-30 minutes. The obtained solid was analyzed by XRD and designated as Gusacitinib Form GS6; as shown in FIG. 6.

### Example 7: Preparation of Gusacitinib-Form GS7

Gusacitinib (Form GS5, 0.1 g) and water (5 mL) were stirred at 25° C. The slurry was stirred at 25° C. for 24 hours, filtered and dried under vacuum at 25° C. for 10-15 minutes. The obtained solid was dried in Air tray dryer at 60° C. for 16 hours, analyzed by XRD and designated as Gusacitinib Form GS7; as shown in FIG. 7.

### Example 8: Preparation of Gusacitinib-Form GS8

Gusacitinib (GS1, 0.350 g) was dissolved in dioxane (8 ml) at 60° C. and filtered. The clear solution was kept for slow solvent evaporation without agitation at 25° C. for 2 days. After 2 days, hexane (3 ml) was added; the reaction mixture was filtered and dried under vacuum for about 30 minutes. The obtained solid was analyzed by XRD and designated as Form GS8 of Gusacitinib; as shown in FIG. 8.

### Example 9: Preparation of Gusacitinib-Form GS9

Gusacitinib (GS3, 0.03 g) was dissolved in N, N-dimethyl formamide (DMF 0.3 ml) at 60° C. To the clear solution, methyl tert-butyl ether (MTBE, 6 ml) was added at 50° C. and maintained for 10 minutes under stirring (mixture remained clear). The reaction mixture was immediately cooled down to −10° C.; and after 10-15 minutes the solution was filtered and dried for 10-15 minutes. The solid was isolated, analyzed by XRD and designated as Form GS9 of Gusacitinib; as shown in FIG. 9.

### Example 10: Preparation of Gusacitinib HCl Salt-Form GHC11

Gusacitinib (Form GS3, 0.05 g) was charged in a glass vial at 25° C. Buffer solution (1.2 pH, 2 ml) was added and the reaction mixture was stirred at 37° C. for 24 hrs. The slurry was filtered and dried under vacuum for 10-15 minutes. The obtained solid was analyzed by XRD and designated as Form GHC11 of Gusacitinib HCl salt; as shown in FIG. 10.

### Example 11: Preparation of Gusacitinib HCl Salt-Form GHC12

Gusacitinib hydrochloride salt (Form GHC11, 5.00 g) was dissolved in methanol (10 ml) at 60° C. To the clear solution, methyl tert-butyl ether (15 ml) was added under stirring at 60° C. The reaction mixture was cooled to 25-30° C. during 15-20 min and maintained for about one hour at this temperature. Reaction mixture was filtered under vacuum for about 15-20 minutes. The obtained solid was dried under vacuum at 25-30° C. for about 30 minutes. The obtained solid was analyzed by XRD and designated as Form GHC12 of Gusacitinib HCl salt; as shown in FIG. 11.

### Example 12: Preparation of Gusacitinib HCl Salt-Form GHC13

A suspension of Gusacitinib (Form GS2, 0.02 g) in isoamyl alcohol (1 ml) was cooled down to 0° C. Hydrochloric acid in isopropyl alcohol (18%, 20 μl) was added at 0° C. and the mixture was stirred about 18 hours at 0° C. The slurry was filtered under vacuum during about 15-20 minutes at room temperature. The obtained solid was analyzed by XRD and designated as Form GHC13 of Gusacitinib HCl salt; as shown in FIG. 12.

### Example 13: Preparation of Gusacitinib HCl Salt-Form GHC14

A suspension of Gusacitinib hydrochloride salt (Form GHC12, 0.02 g) in 3 ml dichloromethane* (3 ml) was heated up to 60° C. and maintained under stirring for about 16 hours. The slurry was cooled down to about 25° C., filtered and dried under vacuum for 10-15 minutes. The obtained solid was analyzed by XRD and designated as Form GHC14 of Gusacitinib HCl salt; as shown in FIG. 13. *Form GHC14 was also obtained when this example was repeated using the following solvents: isobutanol, methanol, ethyl formate, or acetonitrile.

### Example 14: Preparation of Gusacitinib HCl Salt-Form GHC15

Gusacitinib base (Form GS2, 0.2 g) was dissolved in 1,4-Dioxane (10 ml) at 60° C. The reaction mixture was cooled to 8-10° C. during 15-20 minutes and concentrated aqueous HCl (about 37%, 50 μl) was added. The mixture was stirred at 10° C. for about 65 hours. The obtained solid was filtered under vacuum during 30-45 minutes, analyzed by XRD and designated as Form GHC15 of Gusacitinib HCl salt; as shown in FIG. 14.

### Example 15: Preparation of Gusacitinib HCl Salt-Form GHC16

Gusacitinib base (Form GS2, 0.03 g) was dissolved in 1,3 dioxolane (10 ml) at 60° C. The reaction mixture was then cooled to about 0° C. in about 15-20 minutes and concentrated aqueous HCl (about 37%, 20 μl) was added. The reaction mixture was stirred during about 30 hours at about 0° C. and filtered under vacuum for about 15-20 minutes at room temperature. The obtained solid was analyzed by XRD and designated as Form GHC16 of Gusacitinib HCl salt; as shown in FIG. 15.

### Example 16: Preparation of Gusacitinib HCl Salt-Form GHC17

Gusacitinib base (Form GS2, 0.03 g) and 1,2-dichloroethane (2 ml) was mixed together at 25° C. The suspension was cooled to about 10° C. in about 5-10 minutes and hydrochloric acid in isopropyl alcohol (18%, 20 μl) was added. The mixture was stirred for about 18 hours at about 10° C. and filtered under vacuum for about 15-20 minutes at room temperature. The obtained solid was analyzed by XRD and designated as Form GHC17 of Gusacitinib HCl salt; as shown in FIG. 16.

### Example 17: Preparation of Gusacitinib HCl Salt-Form GHC18

Gusacitinib Free base (GS2, 1 g) was suspended in a buffer solution* (pH=1.2, 10 ml) at about 37° C. The slurry was stirred for about 16 hours, cooled to 25° C., filtered and dried under vacuum for about 15 minutes. The solid was further dried in Vacuum tray dryer (VTD) at about 60° C. for 5 hours. The obtained solid was analyzed by XRD and designated as Form GHC18 of Gusacitinib HCl salt; as shown in FIG. 17. *as per USP

### Example 18: Preparation of Gusacitinib HCl Salt-Form GHC18

A mixture of Gusacitinib hydrochloride salt (Form GHC13, 0.25 g) in a buffer solution* (pH=1.2, 8 ml) was heated up to 37° C. The obtained solution was stirred at 37° C. for about 2 hours, filtered and dried under vacuum for 10-15 minutes at room temperature. The obtained solid was further dried in VTD at about 40° C. during 4 hours. The obtained solid was analyzed by XRD-Form GHC18 of Gusacitinib HCl salt. *as per USP

### Example 19: Preparation of Gusacitinib HCl Salt-Form GHC12

Gusacitinib (1 g) was dissolved in 1,3-Dioxolane (30 ml) at 55° C. (clear solution was obtained). Concentrated aqueous HCl (about 37%, 250 μl) was added and the mixture was stirred about 1 hour at 25° C. Reaction mixture was filtered under vacuum for about 15-20 minutes. The solid was isolated and analyzed by XRD-Crystalline Form GHC12 of Gusacitinib HCl salt.

### Example 20: Preparation of Gusacitinib HCl Salt-Form GHC12

A mixture of Gusacitinib (Form GS2, 0.03 g) in THF* (2 ml) was cooled to about 10° C. Hydrochloric acid in isopropyl alcohol (18%, 8 μl) was added and the mixture was stirred about 18 hours at about 10° C. The obtained solid was filtered under vacuum during 15-20 minutes and analyzed by XRD-Crystalline Form GHC12 of Gusacitinib hydrochloride salt. *This example was repeated using different solvents; for example: sulpholane, diacetone alcohol, dimethyl carbonate, dimethyl carbonate, butyl acetate, diethyl ketone, nitromethane, propionitrile, acetone and acetonitrile. Crystalline GHC12 was obtained in all cases.

### Example 21: Preparation of Gusacitinib HCl Salt-Form GHC12

Gusacitinib HCl salt (Form GHC12, 0.02 g) was dissolved in Trifluoroacetic acid (0.2 ml) at 25-30° C. After the solution was filtered, n-butanol* (5 ml) was added at 25-30° C. and the mixture was stirred for about 1 hour. The reaction mixture was maintained under stirring for 2 days and then filtered under vacuum for about 15 minutes. The obtained solid was analyzed by XRD-Form GHC12 of Gusacitinib HCl salt. *This example was repeated with trifluoroacetic acid as a solvent and 1,2-DME, ethyl formate, ethylene glycol, IPA, methyl acetate, methyl isobutyl ketone and toluene as an anti-solvent-Form GHC12 of Gusacitinib HCl salt was always obtained

### Example 22: Preparation of Gusacitinib HCl Salt-Form GHC13

To a mixture of Gusacitinib base (0.05 g) in THF (25 ml) at 0° C., was added an aqueous HCl (about 37%, 0.25 ml). The mixture was stirred about 16 hours at 0° C., filtered under vacuum during about 15-20 minutes and analyzed by XRD-Form GHC13 of Gusacitinib hydrochloride salt.

### Example 23: Preparation of Gusacitinib HCl Salt-Form GHC13

A suspension of Gusacitinib (Form GS2, 0.02 g) in isoamyl alcohol (1 ml) was cooled down to 0° C. HCl (g) in isopropyl alcohol (18%, 20 μl) was added at 0° C. and the mixture was stirred about 18 hours at 0° C. The slurry was filtered under vacuum during about 15-20 minutes at room temperature. The obtained solid was analyzed by XRD and designated as Form GHC13 of Gusacitinib HCl salt (corresponding to FIG. 12).

### Example 24: Preparation of Gusacitinib HCl Salt-Form GHC12

A mixture of Gusacitinib (Form GS2, 0.03 g) in THF* (2 ml) was cooled to about 10° C. HCl (g) in isopropyl alcohol (18%, 8 μl) was added and the mixture was stirred about 18 hours at about 10° C. The obtained solid was filtered under vacuum during 15-20 minutes and analyzed by XRD-Crystalline Form GHC12 of Gusacitinib hydrochloride salt. Crystalline form GHC12 was also formed using the following solvents: sulfolane, diacetone alcohol, dimethyl carbonate, dimethyl carbonate, butyl acetate, diethyl ketone, nitromethane, propionitrile, acetone and acetonitrile.

### Example 25: Stability Experiments

### Storage Stability at Different Relative Humidity

Samples of Gusacitinib Forms: GS2, GS3, GS4 and Gusacitinib HCl salt Forms GHC11, GHC12, GHC18 were subjected to conditions of different relative humidity at ambient temperature. XRPD analysis was performed on the samples after 7 days. The results are shown in Table 1 below:

TABLE 1

| | XRPD analysis results | | |
|---|---|---|---|
| Starting | 60% | 80% | 100% |
| form | (7 days) | (7 days) | (7 days) |
| GS2 | No | No | No |
| | polymorphic | polymorphic | polymorphic |
| | conversion | conversion | conversion |
| GS3 | No | No | No |
| | polymorphic | polymorphic | polymorphic |
| | conversion | conversion | conversion |
| GS4 | No | No | No |
| | polymorphic | polymorphic | polymorphic |
| | conversion | conversion | conversion |
| GHCl1 | No | No | No |
| | polymorphic | polymorphic | polymorphic |
| | conversion | conversion | conversion |
| GHCl2 | No | No | No |
| | polymorphic | polymorphic | polymorphic |
| | conversion | conversion | conversion |
| GHCl8 | No | No | No |
| | polymorphic | polymorphic | polymorphic |
| | conversion | conversion | conversion |

A sample of Form GHC13 of Gusacitinib HCl salt was subjected to 60% relative humidity for 7 days. XRPD analysis revealed no polymorphic conversion.

The above data demonstrates that Forms GS2, GS3 and GS4, and Forms GHC11, GHC12, GHC13, and GHC18 of Gusacitinib HCl salt are especially stable to high relative humidity conditions and are particularly suitable for pharmaceutical dosage forms.

### Grinding Experiments

Samples of Gusacitinib Forms: GS2, GS3, GS4 and Gusacitinib HCl salt Forms: GHC11, GHC12, GHC13, GHC18 were subjected to strong grinding, and to solvent drop grinding in isopropanol, ethanol and water. Grinding was carried out on the sample alone, or in the presence of the solvent. In these experiments, about 20 mg of the sample is placed in a mortar and ground with a pestle for 2 minutes. The solvent, when used, was added to the crystalline material before grinding, in a volume of 10 microlitres. XRPD analysis performed on the sample after the grinding experiment, confirmed no conversion of the starting material (Table 2):

TABLE 2

| | | Gusacitinib HCl salt |
|---|---|---|
| | Gusacitinib | (GHCl1, GHCl2, |
| | (GS2, GS3, GS4) | GHCl3, GHCl8) |
| Condition | XRPD analysis results | |
| Strong grinding | No polymorphic | No polymorphic |
| | conversion | conversion |
| Solvent-drop grinding | No polymorphic | No polymorphic |
| (ethanol) | conversion | conversion |
| Solvent-drop grinding | No polymorphic | No polymorphic |
| (isopropyl alcohol) | conversion | conversion |
| Solvent-drop grinding | No polymorphic | No polymorphic |
| (water) | conversion | conversion |

The results demonstrate that Forms GS2, GS3, GS4 of Gusacitinib, and Forms GHC11, GHC12, GHC13, GHC18 of Gusacitinib HCl salt are resistant to polymorphic changes and are highly suitable for preparing pharmaceutical formulations.

### Thermal Stability

Samples of Gusacitinib Forms: GS2, GS3, GS4 and Gusacitinib HCl salt Forms GHC11, GHC12 and GHC18 were subjected to heating up to 100° C. for 30 minutes. XRPD analysis of the samples confirmed there to be no change in the starting material (Table 3):

TABLE 3

| | Gusacitinib | Gusacitinib HCl salt |
|---|---|---|
| | (GS2, GS3, | (GHCl1, GHCl2, |
| | GS4) | GHCl8) |
| Condition | Result | |
| Heating 100° C., 30 minutes | Stable | Stable |

1. A crystalline Gusacitinib HCl salt.

2. A crystalline form of Gusacitinib HCl salt designated Form GHC11, which is characterized by data selected from at least one of:
(a) an X-ray powder diffraction pattern substantially as depicted in FIG. 10; or
(b) an X-ray powder diffraction pattern having peaks at 5.1, 8.9, 10.1, 15.3 and 27.2 degrees 2-theta±0.2 degrees 2-theta.

3. The crystalline Gusacitinib HCl salt according to claim 2, which is further characterized by an X-ray powder diffraction pattern having any one, two or three additional peaks selected from 17.1, 20.4 and 24.4 degrees 2-theta±0.2 degrees 2-theta.

4. The crystalline Gusacitinib HCl salt according to claim 2, which is characterized by an XRPD pattern having peaks at 5.1, 8.9, 10.1, 15.3, 17.1, 20.4, 24.4, and 27.2 degrees 2-theta±0.2 degrees 2-theta.

5. The crystalline Gusacitinib HCl salt according to claim 2, which is a hydrate.

6. The crystalline Gusacitinib HCl salt according to claim 2, which is substantially free of any other solid state forms of Gusacitinib and/or Gusacitinib salt.

7. A crystalline form of Gusacitinib HCl salt designated Form GHC12, which is characterized by data selected from at least one of:
(a) an X-ray powder diffraction pattern substantially as depicted in FIG. 11; or
(b) an X-ray powder diffraction pattern having peaks at 6.4, 13.7, 15.6, 22.9 and 27.5 degrees 2-theta±0.2 degrees 2-theta.

8. The crystalline Gusacitinib HCl salt according to claim 7, which is further characterized by an X-ray powder diffraction pattern having any one, two, three or four additional peaks selected from 12.7, 14.4, 16.6 and 17.8 degrees 2-theta±0.2 degrees 2-theta.

9. The crystalline Gusacitinib HCl salt according to claim 7, which is characterized by an XRPD pattern having peaks at 6.4, 12.7, 13.7, 14.4, 15.6, 16.6, 17.8, 22.9 and 27.5 degrees 2-theta±0.2 degrees 2-theta.

10. The crystalline Gusacitinib HCl salt according to claim 7, which is anhydrous.

11. The crystalline Gusacitinib HCl salt according to claim 7, which is substantially free of any other solid state forms of Gusacitinib and/or Gusacitinib salt.

12. A crystalline form of Gusacitinib HCl salt designated Form GHC13, which is characterized by data selected from at least one of:
(a) an X-ray powder diffraction pattern substantially as depicted in FIG. 12; or
(b) an X-ray powder diffraction pattern having peaks at 5.5, 16.7, 21.0, 22.0 and 26.2 degrees 2-theta±0.2 degrees 2-theta.

13. The crystalline Gusacitinib HCl salt according to claim 12, which is further characterized by an X-ray powder diffraction pattern having any one, two, three or four additional peaks selected from 14.5, 19.0, 24.8 and 28.6 degrees 2-theta±0.2 degrees 2-theta.

14. The crystalline Gusacitinib HCl salt according to claim 12, which is characterized by an XRPD pattern having peaks at 5.5, 14.5, 16.7, 19.0, 21.0, 22.0, 24.8, 26.2 and 28.6 degrees 2-theta±0.2 degrees 2-theta.

15. The crystalline Gusacitinib HCl salt according to claim 12, which is a hydrate.

16. The crystalline Gusacitinib HCl salt according to claim 12, which is substantially free of any other solid state forms of Gusacitinib and/or Gusacitinib salt.

17. A crystalline form of Gusacitinib HCl salt designated Form GHC14, which is characterized by data selected from at least one of:
(a) an X-ray powder diffraction pattern substantially as depicted in FIG. 13; or
(b) an X-ray powder diffraction pattern having peaks at 5.8, 8.0, 14.9, 21.5 and 25.4 degrees 2-theta±0.2 degrees 2-theta.

18. The crystalline Gusacitinib HCl salt according to claim 17, which is further characterized by an X-ray powder diffraction pattern having any one, two, three or four additional peaks selected from 9.4, 12.7, 15.6, 17.8 and 22.9 degrees 2-theta±0.2 degrees 2-theta.

19. The crystalline Gusacitinib HCl salt according to claim 17, which is characterized by an XRPD pattern having peaks 5.8, 8.0, 9.4, 12.7, 14.9, 15.6, 17.8, 21.5, 22.9 and 25.4 degrees 2-theta±0.2 degrees 2-theta.

20. The crystalline Gusacitinib HCl salt according to claim 17, which is anhydrous.

21. The crystalline Gusacitinib HCl salt according to claim 17, which is substantially free of any other solid state forms of Gusacitinib and/or Gusacitinib salt.

22. A crystalline form of Gusacitinib HCl salt designated Form GHC18, which is characterized by data selected from at least one of:
(a) an X-ray powder diffraction pattern substantially as depicted in FIG. 17; or
(b) an X-ray powder diffraction pattern having peaks at 5.8, 11.4, 17.1, 24.6 and 27.5 degrees 2-theta±0.2 degrees 2-theta.

23. The crystalline Gusacitinib HCl salt according to claim 22, which is further characterized by an X-ray powder diffraction pattern having any one, or two additional peaks selected from 9.0 and 28.6 degrees 2-theta±0.2 degrees 2-theta.

24. The crystalline Gusacitinib HCl salt according to claim 22, which is characterized by an XRPD pattern having peaks at 5.8, 9.0, 11.4, 17.1, 24.6, 27.5 and 28.6 degrees 2-theta±0.2 degrees 2-theta.

25. The crystalline Gusacitinib HCl salt according to claim 22, which contains water in an amount of: about 5.0 to about 10.0 wt %; about 5.5 to about 9.5 wt %, or about 6.0 to about 9.0 wt %, preferably which is a hydrate; and more preferably a dihydrate.

26. The crystalline Gusacitinib HCl salt according to claim 22, which is substantially free of any other solid state forms of Gusacitinib and/or Gusacitinib salt.

27-36. (canceled)

37. A method for preparing other crystalline forms of Gusacitinib, salts of Gusacitinib or crystalline forms thereof using the crystalline Gusacitinib HCl salt of claim 1.

38. (canceled)

39. A pharmaceutical composition comprising crystalline Gusacitinib HCl salt according to claim 1, and at least one pharmaceutically acceptable excipient.

40. A process for preparation of a pharmaceutical composition and/or formulation using the crystalline Gusacitinib HCl salt of claim 1.

41. A process for preparing a pharmaceutical composition comprising the crystalline Gusacitinib HCl salt according to claim 1, the method comprising combining the crystalline Gusacitinib HCl salt according to claim 1 with at least one pharmaceutically acceptable excipient.

42. A method for treating a condition, comprising administering a therapeutically effective amount of crystalline Gusacitinib HCl salt according to claim 1 as a medicament to a subject in need of the treatment.

43. A method of treating a condition, comprising administering a therapeutically effective amount of crystalline Gusacitinib HCl salt according to claim 1 in the treatment of chronic hand eczema, or moderate to severe atopic dermatitis.

44. A method of treating tumors comprising administering a therapeutically effective amount of crystalline Gusacitinib HCl according to claim 1, to a subject in need of the treatment.
